(19) United States
(12) Patent Application Publication
(10) Pub. No.: US 2026/0248997 A1
(43) Pub. Date: Aug. 27, 2026

(54) A DEVICE FOR IN SITU INFLATABLE VACUUM-ASSISTED FISTULA-THERAPY

(71) Applicants: Kamal DAAS, Ramat Gan (IL); MOR RESEARCH APPLICATIONS LTD., Ramat Gan (IL)

(72) Inventors: Kamal DAAS, Ramat Gan (IL); Aharon AMIR, Ramat Gan (IL); Muhammad MANSOUR, Ramat Gan (IL)

(21) Appl. No.: 18/994,050

(22) PCT Filed: Jul. 13, 2023
(86) PCT No.: PCT/IL2023/050730

§ 371 Date: Jan. 13, 2025

(30) Foreign Application Priority Data
Jul. 14, 2022 (IL) 294784

(51) Int. Cl. A61M 1/00; A61M 3/02; A61M 25/10

(52) U.S. Cl. CPC A61M 1/92; A61M 3/0295; A61M 2025/1013; A61M 2025/105

(57) ABSTRACT

The present disclosure discloses a device for in situ endoscopic vacuum-assisted fistula-therapy having a distal portion locatable within the fistula and interconnectable proximal portion. The device of the present disclosure comprising at least one expandable unit being configured and operable to be inserted and advanced into a fistula, comprising (i) and inflatable balloon; (ii) an inner core located within the inflatable balloon; (iii) an outer expandable envelope at least partially surrounding said inflatable balloon; and (iv) at least one vacuum tube being disposed in between an outer surface of the inflatable balloon and the outer expandable envelope. The outer envelope is at least partially made of a porous material configured to allow passage of fluids from the fistula towards said at least one vacuum tube. Furthermore, said at least one vacuum tube is configured and operable to be connectable to a vacuum source to drain fluids having passed through the outer expandable envelope by applying a negative pressure.

### TECHNOLOGICAL FIELD

The present disclosure relates to a device for in situ inflatable vacuum-assisted fistula-therapy.

### BACKGROUND ART

References considered to be relevant as background to the presently disclosed subject matter are listed below:
EP 3,506,956
US 2018/264,239
EP 2,854,893

Acknowledgement of the above references herein is not to be inferred as meaning that these are in any way relevant to the patentability of the presently disclosed subject matter.

### BACKGROUND

A fistula is an abnormal connection between two hollow spaces, typically between two epithelialized surfaces, such as blood vessels, intestines, or other hollow organs. Fistulas can arise from various causes, including spontaneous occurrences (such as diverticulitis or Crohn's disease), traumatic injuries, iatrogenic factors (related to surgery or diagnostic procedures), radiation, malignancy, or congenital conditions. The classification of fistulas can be based on their specific location.

An enterocutaneous fistula (ECF) is an abnormal connection that develops between the intestinal tract or stomach and the skin, leading to the leakage of the stomach or intestinal contents onto the skin surface. ECFs are commonly observed as a complication following bowel surgery. ECFs occurring after an emergency general surgery for gastrointestinal conditions are associated with a notable rise in the likelihood of mortality and readmission, with the rates continuing to increase for at least 90 days post-surgery.

Women may develop an abnormal connection between the large intestine and vagina due to factors such as childbirth, disease, or complications arising from surgery. This condition, known as a rectovaginal fistula, can lead to the leakage of bowel contents into the vagina, posing a risk of infection for the patient.

Fistula complications and implications encompass a range of significant factors, including malnutrition, anemia, and electrolyte imbalances. Hemorrhage and hematoma may occur, as well as the risk of infection, skin inflammation, abscess formation, sepsis, and even death. Individuals with fistulas often experience challenges related to ongoing care, discomfort, unpleasant odors, and feelings of isolation. The presence of a fistula can contribute to a depressive state, while its management necessitates long-term care, reoperations, and readmissions, resulting in substantial healthcare costs.

The current approaches and challenges in managing fistulas can be summarized as follows: Conservative treatment options include the use of antibiotics and anti-inflammatory medications, along with wound care practices, suspension of oral feeding, the application of plugs, fibrin glue, or stents. Surgical interventions involve techniques such as excision with healing by secondary intention, primary closure, local flap procedures, or the use of free flaps. However, these approaches come with significant challenges, primarily associated with the risk of recurrence and the added complexity they may introduce to the treatment process.

The endoscopic vacuum-assisted closure system is a method used to manage anastomotic leaks and perforations. Vacuum-assisted therapy has gained wide application in the treatment of skin and muscular defects. By applying suction, it helps to reduce wound secretion and edema, improve microcirculation, promote granulation of the wound, and facilitate wound size reduction through retraction. The use of Vacuum-assisted closure (VAC)-endoscopic application for perforations and fistulas has been discussed in the literature, as demonstrated by the successful case series and literature review conducted by Rubicondo et al. in their publication titled “Endoluminal vacuum-assisted closure (E-Vac) therapy for postoperative esophageal fistula: successful case series and literature review” in the World Journal of Surgical Oncology, 18.1 (2020): 1-7.

The Endo-Sponge® kit, commercially available from B. Braun Surgical, S. A. (ES), is priced at approximately £250 (excluding VAT) for a single sponge. It has been estimated that a complete treatment with the kit would typically necessitate the use of 7 to 8 sponges, resulting in a total cost of around £2,000. Additionally, the Redyrob®TransPlus®bottle, used in conjunction with the kit, is purchased separately and costs approximately £21 per bottle (excluding VAT). In addition to the high cost, there are several other disadvantages associated with the device. These include the use of a single sponge size, which can create shear forces and require lubrication. Furthermore, the one-size-fits-all sponge may not adequately accommodate different fistula widths, and it cannot be adjusted in size as the fistula heals. Additionally, the device cannot be effectively integrated with an endoscope, and there is no plug available for effective sealing.

Similar limitations and challenges apply to treatment of a sinus wound, which is a discharging blind-ended track that extends from the surface of an organ to an underlying area or abscess cavity.

### GENERAL DESCRIPTION

Therefore, there is still a significant need for a cost-effective tool for vacuum-assisted closure VAC-endoscopic conservative treatment for treating fistula and/or sinus wounds. This tool should be capable of actively draining fluids to prevent infection, while also being adaptable to fit fistulas of various sizes and dimensions in terms of both length and width. Ideally, the device should allow for gradual removal as the fistula heals and shrinks, without generating shear force during insertion owing to the inflatable balloon being deflated. Furthermore, it should enable delivery and usage over an endoscope. Fulfilling these requirements would address a long-standing need in this field.

One object of the disclosure is to disclose a device for in situ endoscopic vacuum-assisted fistula-therapy having a distal portion locatable within the fistula and interconnectable proximal portion. The device of the present disclosure comprising at least one expandable unit being configured and operable to be inserted and advanced into a fistula or sinus wound, comprises (i) an inflatable balloon; (ii) an inner core located within the inflatable balloon; (iii) an outer expandable envelope at least partially surrounding said inflatable balloon; and (iv) at least one vacuum tube being disposed in between an outer surface of said inflatable balloon and the outer expandable envelope. The outer envelope is at least partially made of a porous material and is configured to allow passage of fluids from the fistula cavity towards said at least one vacuum tube. Furthermore, said at least one vacuum tube is configured and operable to be connectable to a vacuum source to drain fluids having passed through the outer expandable envelope by applying a negative pressure.

The term “fistula” is to be understood as any wound diagnosed as a fistula of all types of categories, pathophysiology, severities, locale, etc. An object of the present disclosure is to provide a device for in situ endoscopic vacuum-assisted fistula-therapy, which may also be used for treating sinus wounds. Therefore, the term “fistula” is to be understood as further relating to sinus wounds. The fistula cavity refers to the hollow spaced lined with tissue (i.e., endoluminal surface) formed between two organs or between an organ and the skin surface. The fistula dimensions refer to the size and extent of a fistula. The dimensions of a fistula may include length, i.e., the extent of the fistula tract from its origin to its endpoint, the diameter, i.e., the width or thickness of the fistula tract at a specific point, the depth indicating how far the fistula extends into the surrounding tissues or organs, etc. The inner shape of the fistula refers to the internal geometry or structure of the fistula.

The term “inner core” defines hereinafter a portion of the device disclosed herein that is located substantially at the center of the device. It may further comprise specifically, albeit not exclusively, the center of an at least one expandable unit comprising the device. It may further be defined by a hollow volume being enclosed by an open-bore sheath accommodated within. The term “porous material” refers to a solid material that contains interconnected voids, pores, or open spaces within its structure. These pores can range in size and shape, and they may be interconnected or isolated from one another. The presence of these pores gives the material its porous nature, allowing for the passage or absorption of fluids, gases, or other substances. In a specific and non-limiting embodiment, the porous material may be a flexible biocompatible polymeric foam, at least partially made of, for example, polyurethane foam (PUF) composites, such as PUF-graphene oxide (GO), PUF-polypyrrole (PPy)-GO, and PUFpoly (3,4-ethylenedioxythiophene) (PEDOT)-GO); polymeric scaffold having interconnected pores and comprising a plurality of cross-linked star block copolymers, each star block copolymer having a plurality of pendant liquid crystal side chains, such as e.g., 2,2-Bis(1-caprolactone-4-yl) propane (BCP) and derivatives thereof, and Bis-caprolactone with oligoethylene glycol spacer and derivatives thereof.

In yet another non-limiting embodiment of the present disclosure, the outer expandable envelope may be further at least partially coated, enveloped by, doped with, or otherwise comprises, at least one material being capable of at least one of accelerating healing or treating an infection. The at least one material may comprise one or more additives and/or active materials, including, inter alia, drugs and compositions of pharmaceutical local activity; biocides such as inorganic metals, e.g., silver, or organic compositions; sclerosing agents, such as sodium tetradecyl sulfate, polidocanol, ethanolamine oleate, and any mixture and combinations thereof. At least a portion of those materials may be configured to be activated in situ, for a slow, sustained release of the active material.

According to another non-limiting embodiment of the present disclosure, the porous material in itself can also be selected to be capable of at least one of accelerating healing or treating an infection. The porous material may be at least partially coated, enveloped by, doped with, or otherwise comprises, at least one material (medicine or another active substance) including one or more additives and/or active materials, including, inter alia, drugs and compositions of pharmaceutical local activity; biocides such as inorganic metals, e.g., silver, or organic compositions; sclerosing agents, such as sodium tetradecyl sulfate, polidocanol, ethanolamine oleate, and any mixture and combinations thereof. At least a portion of those materials may be configured to be activated in situ, for a slow, sustained release of the active material.

According to yet another embodiment of the present disclosure, the inner core comprises at least one first open-bore sheath configured to at least one of accommodating an endoscope tube or inflating tube being couplable with the inflatable balloon and being configured and operable to at least partially inflate and deflate said inflatable balloon in situ. The term “vacuum” refers to a state or condition of negative pressure, which in an aspect of this disclosure may encompass a range of about 20 mmHg to about 700 mmHg, e.g., 115 mmHg. It is therefore to be understood that the terms “vacuum” and “negative pressure” are used interchangeably to imply the same meaning. To achieve effective vacuum along the device, the distal end portion of the device is optionally configured to be sealed by a sealing element (e.g., plug).

Another object of the disclosure is to disclose a device for in situ endoscopic vacuum-assisted fistula-therapy as defined above, wherein the device is configured to be inserted into the fistula while the inflatable balloon is in a deflated state, such that no shear force is applied on an endoluminal surface of the fistula. Another object of the disclosure is to disclose a device as defined in any of the above, wherein the inflatable balloon is configured to expand the outer expandable envelope when at least partially inflated, thereby pressing an outer surface of the outer expandable envelope to contact an endoluminal surface of the fistula.

Another object of the present disclosure is to disclose a device as defined in any of the above, wherein the at least one vacuum tube is disposed on an outer surface of the inflatable balloon, and further has flexible properties, such that the outer shape of the at least one vacuum tube corresponds to the shape of the outer surface of the inflatable balloon. In accordance with a non-limiting embodiment of the present disclosure, the device further comprises at least one vacuum suction port being configured for connecting between a plurality of vacuum tubes of the same expandable unit.

Another object of the disclosure is to disclose a device for in situ endoscopic vacuum-assisted fistula-therapy as defined in any of the above, wherein the device comprises at least one connection port being configured for connecting at least one expandable unit to another expandable unit provided in series, thereby, when connected, permitting fluid communication between consecutive expandable units. In accordance with a non-limiting embodiment of the present disclosure, the at least one connection port is configured to allow an angular orientation of at least one expandable unit with respect to another expandable unit, such that an outer shape of the plurality of expandable units comprising the device, when connected, corresponds to at least one of the dimensions and inner shape of the fistula. In accordance with yet another non-limiting embodiment of the present disclosure, the at least one connection port is configured to further accommodate at least one of endoscopic tube or inflation tube being configured and operable to at least partially in situ inflate or deflate an inflatable balloon of any of the at least one expandable unit.

Another object of the disclosure is to disclose the use of the device for in situ endoscopic vacuum-assisted fistula-therapy, as defined in any of the above. The use comprises providing a device for in situ inflatable vacuum-assisted fistula-therapy as defined above. If required, the use comprises inserting an endoscope at a location at the distalmost portion of the device via an endoscope inlet being connectable to the at least one first open-bore sheath. The use further comprises advancing the device towards a fistula to be treated; in situ at least partially inflating the inflatable balloon, and; applying vacuum thereby draining fluids from the fistula that pass through the outer expandable envelope towards the at least one vacuum tube.

In a preferred non-limiting embodiment of the device disclosed herein, each of the at least one expandible unit may be configured to be selectively at least partially in situ inflated or deflated independently one to the other to different dimensions, i.e., different volumes, to provide an overall external shape of the device corresponding to an internal shape of fistulas of various sizes and dimensions. In this way, when inflated, the device for in situ endoscopic vacuum-assisted fistula-therapy, as defined in any of the above, may be permitted to effectively contact as much of the endoluminal surface of the fistula as possible. Moreover, to ensure a modular and dynamic fit, each of the at least one expandable unit may be further configured to be selectively, discretely inflated or deflated; also being further configured to be sequentially at least partially inflated with respect to another expandable unit being placed proximally to form a distal portion having an adjustable length being configured to be deployed in fistulas of different dimensions.

In accordance with some embodiments of the present disclosure, the device is further configured to permit the gradual withdrawal of the at least one expandable unit to facilitate gradual healing of the wound from the deeper, distal direction to the more superficial, proximal direction; thereby improving healing processes of the fistula by preventing the fistula wound from healing and closing in the superficial direction, while an active wound remains in the depth of the tissue. To achieve this, each expandable unit is configured to be sequentially deflated with respect to the expandable unit being placed distally to allow gradual removal of the device as the fistula heals and shrinks.

In accordance with some embodiments of the present disclosure, each of the at least one expandable unit may comprise an inflatable balloon, wherein the inflatable balloon may have different shapes, such as narrow cylinders, elliptical, wider cylinders, or a combination of cylinders of heterogeneous diameter, narrow in portions relatively close to the proximal end, and wider at the distal end.

The use as defined above also comprises the step of applying vacuum in either a constant or variable manner over time.

Additionally, or alternatively, the use as defined above may comprise steps of applying a train of pulses over time. The pulses may be trains of both (i) pulses of negative pressure being either constant or variable; and (ii) at least one positive pressure. The application of such pulses enables to prevent from the outer expandable envelope, and consequently the device to be blocked by fluids of varying viscosity (gel-like non-or less-flowing substances), or solids (blood clots) drained from the fistula and to advance the device progressively in the fistula by operating a series of pulses.

Another object of the disclosure is to disclose a device for in situ endoscopic vacuum-assisted fistula-therapy, as defined in any of the above, wherein the inner core may further comprise at least one insertion channel, being configured and operable for at least one of temporary insertion, manipulation, or activation of at least one of (i) at least one surgical tool, or (ii) an immobilizer being configured and operable to attach the device to the fistula. The number of insertion channels is not limited and may be unified, hybrid, or otherwise incorporated together into a single (one), two, three, four, or five different channels. In some embodiments of the present disclosure, the use further comprises inserting and manipulating surgical tools via the at least one open-bore sheath. The surgical tools may be selected from at least one of manipulators, light, heat or electricity emitters, sensors, obstetrical forceps, trocar incision closure devices, electrodes and electrosurgical cables, cannulas and trocars, laparoscopic bipolar scissors and graspers, forceps and graspers, hooks and probes, knot pushers, needles and needle holders, retractors, scissors, suturing tools, probes, dissectors, or any combination thereof. The immobilizer as used herein, is selected from an anchoring means, locators, fixating means, sutures, patch, clamp(s), biocompatible adhesives, etc., including those shaped as inflatable balloons, or made of shape memory alloys (e.g., nitinol), anchors or studs of changeable (open-close, thin-wide) configuration. Additionally, or alternatively, the inner core may comprise at least one drug delivery tube being configured and operable to allow passage of the drug therethrough and to administer at least one drug in a proximal to distal direction of the device.

Another object of the disclosure is to disclose an in situ vacuum-assisted fistula-therapy device, as defined in any of above, further comprising a control unit being connectable to a pressure source and being configured and operable to operate the device by applying vacuum in a constant and/or variable manner. Additionally, or alternatively, the inner core, as defined by any of the above, may comprise at least one drug delivery tube being configured and operable to allow passage of a drug therethrough and to administer at least one drug in a proximal to distal direction of the device. In accordance with some embodiments of present disclosure, the control unit may be further connectable to a drug reservoir and may be configured and operable to administer a drug by applying a plurality of pulses of positive pressure e.g., via the drug delivery tube.

The use as defined above may also comprise the administration of a drug by applying a positive pressure as a part of an operational scheme, which includes applying a train of pulses of both (i) pulses of negative pressure for draining fluids from the fistula, being either constant and/or variable; and then, periodically, (ii) applying at least one positive pressure, hence administrating the drug.

As used herein, the term “substantially” refers to the complete or nearly complete extent or degree of an action, characteristic, property, state, structure, item, or result.

As used herein the term “about” refers to plus or minus 10 percent.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C represent schematical, out-of-scale illustrations of a possible configuration of the device of the present disclosure; in particular, FIG. 1A provides a sideview, FIG. 1B provides a perspective view showing a cross section of the expandable unit's distal portion, and FIG. 1C provides a cross-sectional view of the expandable unit's proximal portion;

FIGS. 2A-2B represent schematical sideview illustrations of the inner portion of an at least one expandable unit, without depicting the expandable outer envelope, wherein the inflatable balloon is either in a deflated state (FIG. 2A) or inflated state (FIG. 2B).

FIGS. 3A-3D represent different possible configurations of the device of the present disclosure comprising more than one expandable unit; in particular wherein: FIGS. 3A-3B represent the device in the deflated and inflated state, respectively; FIG. 3C represents a differential inflation operational mode; and FIG. 3D represents a possible deployment of the device within a fistula showing possible differential inflation; and

FIGS. 4A-4D provide schematic illustrations of different possible operational modes of the device according to some embodiments of the present disclosure.

### DETAILED DESCRIPTION OF EMBODIMENTS

In the figures and descriptions set forth, identical reference numerals indicate those components that are common to different embodiments or configurations of the device disclosed herein.

Further, it will be appreciated that for simplicity and clarity of illustration, the figures are schematic, and elements shown in the figures have not necessarily been drawn to scale. For example, the dimensions of some of the elements may be exaggerated relative to other elements for clarity.

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

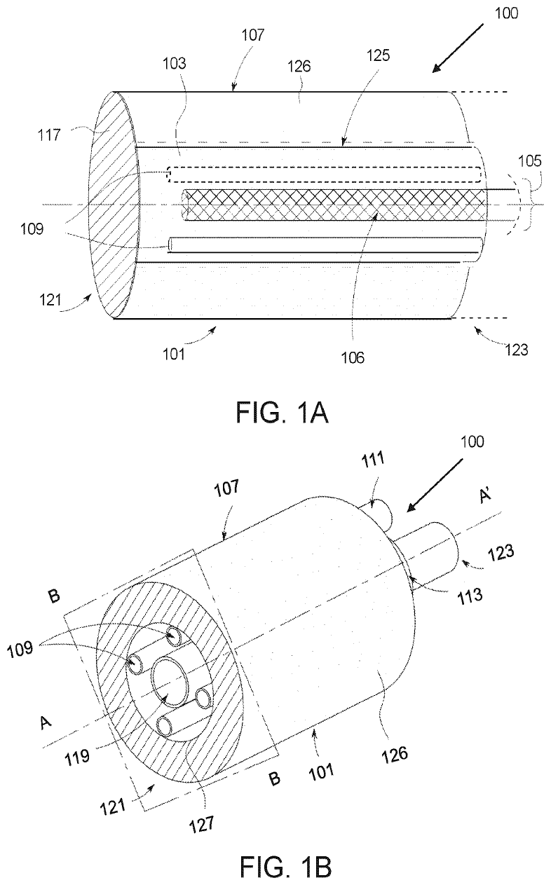

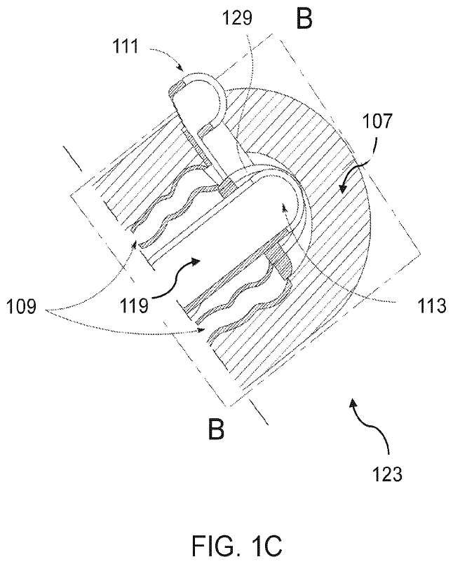

Reference is made to FIGS. 1A-1C, providing schematic, out-of-scale illustrations of a possible device for in situ endoscopic vacuum-assisted fistula-therapy, in accordance with several non-limiting embodiments of the present disclosure. FIG. 1A provides a side view, with a sideview of the elements positioned within the outer expandable envelope demarcated by dashed lines. Device 100 comprises at least one expandable unit 101 being configured and operable to be inserted into a fistula. The at least one expandable unit 101 comprises an inflatable balloon 103 which may have a predefined shape, an inner core 105 located within the inflatable balloon 103, an outer expandable envelope 107 at least partially surrounding inflatable balloon 103, and at least one vacuum tube 109 being disposed in between an outer surface of inflatable balloon 103 and the outer expandable envelope 107, wherein said at least one vacuum tube is configured and operable to be connectable to a vacuum source to drain fluids having passed through the outer expandable envelope by applying a negative pressure. The at least one expandable unit 101 may be configured and operable to be inserted into the fistula while the inflatable balloon is in a deflated state, such that no shear force is applied on the endoluminal surface.

Device 100 also comprises, inter alia, a distal portion 121 locatable within a fistula to be treated and its interconnectable proximal portion 123. The distal portion 121 may comprise at least one in situ inflatable balloon 103 and at least one inflation tube 106 being configured and operable to at least partially in situ inflate or deflate inflatable balloon 103. FIG. 1B provides a perspective view with cross section of a device 100 comprising at least one expandable unit 101. Preferably, device 100 has a main longitudinal axis A: A′, and a predefined cross section B, wherein cross section B is oriented perpendicular to the main axis A: A'. The at least one expandable unit 101 may comprise an inflatable balloon 103, an inner core 105 located within the inflatable balloon 103 provided in parallel with axis A: A′, an outer expandable envelope 107 at least partially surrounding the inflatable balloon 103, and at least one of vacuum tube 109 being configured to drain fluids and being disposed in between an outer surface 125 of the inflatable balloon 103 and an inner surface 127 of the outer expandable envelope 107. Outer expandable envelope 107 may be at least partially made of a porous material such as a biocompatible polymeric foam and is configured to allow the passage of drained fluids from the outmost surface of the balloon 103, namely from the fistula cavity, in the direction of the inner core 105, towards at least one vacuum tube 109 when negative pressure is being applied. When at least partially inflated, the inflatable balloon 103 is configured to expand the outer expandable envelope 107 of the at least one expandable unit 101. The inflatable balloon, when at least partially inflated, may be configured to permit pressing an outer surface 126 of the outer expandable envelope 107 to contact an endoluminal surface of the fistula.

In some embodiments, device 100 comprises a plurality of vacuum tubes 109 being arranged in parallel with axis A: A′, when the inflatable balloon 103 is in a deflated state. Each of the plurality of vacuum tubes 109, which may be disposed on outer surface 125 of the inflatable balloon 103, is configured and operable to be connected to a vacuum source (not shown) through a vacuum suction port 111 to drain primarily fluids from the fistula cavity, but also from the fistula environment, having passed through the outer expandable envelope by applying a negative pressure.

In some embodiments, inner core 105 comprises at least one open-bore sheath 119 being configured to be accommodated within inner core 105. The at least one open-bore sheath 119 may be configured for at least temporarily or reversibly accommodating an endoscope tube and/or an inflating tube 106 being couplable with the inflatable balloon 103 and being configured and operable to at least partially inflate and deflate inflatable balloon 103.

The elements of device 100 may be at least partially made of biocompatible materials, including polyethylene, polyolefins, polyvinyl chloride, polyester, polyimide, polyetherketone, polyethylene terephthalate (PET), polyamides, nylon, polyurethane, derivatives, blends, copolymers thereof, and the like. In a specific and non-limiting embodiment of the device disclosed herein, the outer expandable envelope 107 of the at least one expandable unit 101 may be a biocompatible polymeric foam, at least partially made of, for example, polyurethane foam (PUF) composites, such as PUF-graphene oxide (GO), PUF-polypyrrole (PPy)-GO, and PUFpoly (3,4-ethylenedioxythiophene) (PEDOT)-GO); polymeric scaffold having interconnected pores and comprising a plurality of cross-linked star block copolymers, each star block copolymer having a plurality of pendant liquid crystal side chains, such as e.g., 2,2-Bis(1-caprolactone-4-yl) propane (BCP) and derivatives thereof, and Bis-caprolactone with oligoethylene glycol spacer and derivatives thereof.

In accordance with some embodiments of the present disclosure, device 100 may optionally comprise a sealing component, e.g., a plug 117, at its distal end portion 121, for maintaining effective pressure throughout the device 100 when the vacuum source is turned on and preventing excessive drainage of fluids from a site external to the fistula, for example, from the intestines in the case of an enterocutaneous fistula (ECF).

As can be appreciated in FIG. 1C, the proximal portion 123 of expandable unit 101, shown along cross section B, comprises an endoscope inlet 113 and a vacuum suction port 111 being connectable to a vacuum source (not shown) and to the possible plurality of vacuum tubes 109 at their distal end along axis A: A′ through connector element 129 for connecting between a plurality of vacuum tubes of the same expandable unit, if any.

The total length of device 100 ranges from about 50 mm to about 500 mm. The outer diameter may range from about 0.5 mm to about 15 mm, in particular about 1 mm to about 5 mm, preferably about 1, 2 or 3 mm. In another embodiment of device 100 disclosed herein, the at least one expandable unit 101 may have a length in the range of about 10 mm to 100 mm. Open-bore sheath 119 may be in fluid communication with the inflatable balloon 103 and may be used to at least partially inflate or deflate the inflatable balloon 103. Alternatively, the inner diameter of the open-bore sheath 119 may be positioned within the inflatable balloon 103 and may be able to accommodate at least an inflation tube or endoscope tube. The inner diameter of open-bore sheath 119 may be in a range of about 0.1 mm to 13 mm, in particular about 0.2 mm to about 4 mm, preferably about 0.5 mm to about 2 mm.

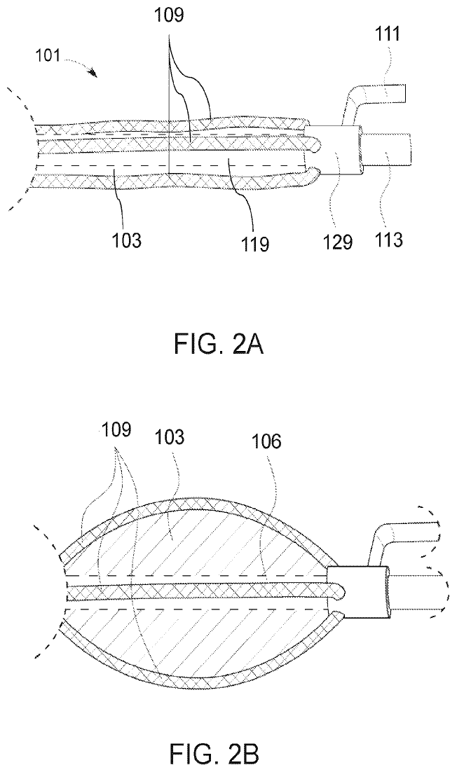

Reference is made to FIGS. 2A-2B, which provide schematic side views of an at least one expandable unit 101, in accordance with a non-limiting embodiment of the present disclosure, wherein the inflatable balloon 103 provided along main axis A: A′ is at least partially deflated (FIG. 2A) or inflated (FIG. 2B). For illustrative purposes, the outer expandable envelope of the expandable unit 101 has been omitted to permit a clear representation of the inflatable balloon 103 in its different states. As can be appreciated, in some embodiments, in the deflated state being used for deployment of the device in the fistula for avoiding shear force being applied on the endoluminal surface, the plurality of vacuum tubes 109 are provided in parallel to axis A: A′ and are connectable to a vacuum suction port 111 at the distal end portion 121 through connector element 129. In accordance with some embodiments of the present disclosure, each of the plurality of vacuum tubes has flexible properties, such that the outer shape of a vacuum tube 109 corresponds to the shape of the outer surface of the balloon when it is at least partially inflated or deflated.

As disclosed above with respect to FIGS. 1A-1B, device 100 comprises at least one inflation tube 106 or open-bore sheath 119, comprising an endoscope tube in accordance with an embodiment of the present disclosure, being configured and operable to at least partially in situ inflate or deflate inflatable balloon 103. The at least partial inflation of the inflatable balloon 103, in accordance with some embodiments of the present disclosure, is facilitated by passing/applying a fluid (e.g., saline, purified water, CO2 gas, etc.) through the inflation tube or endoscope tube, optionally accommodated within the open-bore sheath 119, in a predefined volume, rate, and pressure, so that inflatable balloon 103 is shaped and having the corresponding dimensions of the fistula and/or of an area adjacent to the fistula.

In one embodiment, inflatable balloon 103, when at least partially inflated, directly shapes the outer expandable envelope 107, and consequently the expandable unit 101. In another embodiment of the present disclosure, only predefined portions of the outer surface of the inflatable balloon 103 are actively inflated. To achieve this, inflatable balloon 103 may be covered by a net of inflating/deflating tubes (not shown) wherein inflating fluid is passed/applied. Portions in between the array of the fluid channels are inflated to a lesser extent, such that inflatable balloon 103 is not evenly inflated.

In accordance with several embodiments of the present disclosure, the inflatable balloon 103 and/or outer expandable envelope 107 may be further at least partially coated, enveloped by, doped with, or otherwise comprises, one or more additives and/or active materials, including, inter alia, drugs and compositions of pharmaceutical local activity; biocides such as inorganic metals, e.g., silver, or organic compositions; sclerosing agents, such as sodium tetradecyl sulfate, polidocanol, ethanolamine oleate, and any mixture and combinations thereof. At least a portion of those materials may be configured to be activated in situ, for a slow, sustained release of the active material.

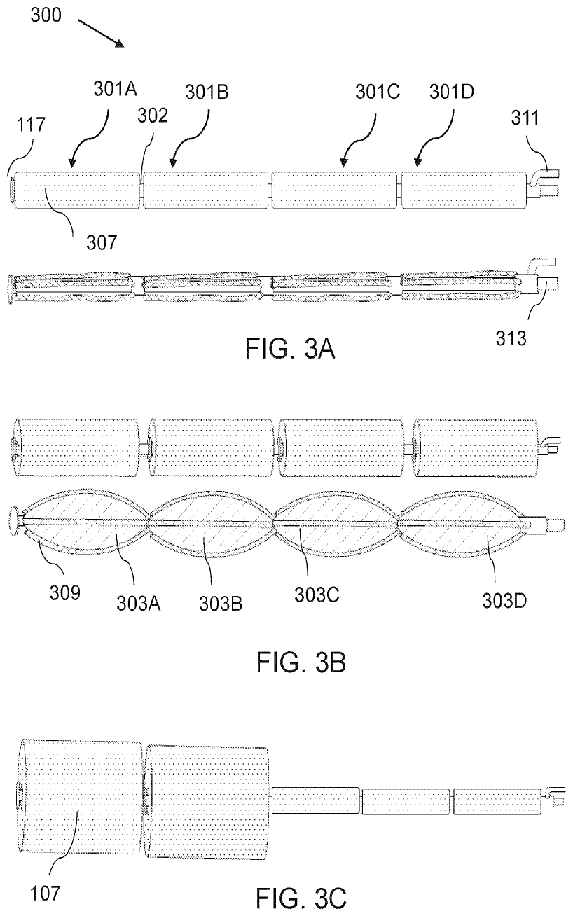

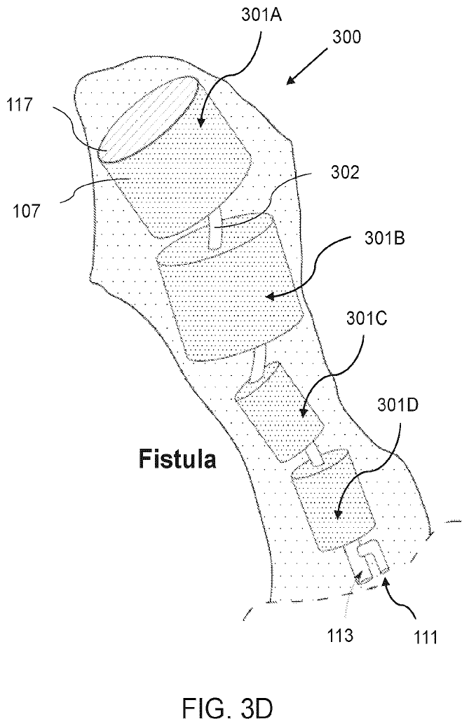

Reference will now be made to FIGS. 3A-3C, which illustrate several non-limiting embodiments of a device 300 for in situ endoscopic vacuum-assisted fistula-therapy comprising a plurality of expandable units 301A-301D, as previously described, connected in series; in a manner reminiscent of the organization of the earthworm's (phylum Annelida) “rings” or annuli. FIGS. 3A-3B provide a schematic sideview illustration of a series of expandable units 301A-301D, wherein all expandable units 301A-301D are concurrently either in a deflated or inflated state, respectively. Although the figures show four expandable units, this number was selected randomly for demonstrative and illustrative purposes only, and the configuration of device 300 is strictly not limited to any number of expandable units. Any number of units may be selected and configured in any relevant arrangement to accommodate a variety of fistulas, in terms of shape, dimensions, wound type, wound severity, physical/anatomical locale of the fistula, etc. As can be understood from FIGS. 3A-3B, the distal most expandable unit 301A may comprise an optional plug 117 at its distal end portion so that a vacuum is effectively applied upon outer expandable envelope 307 when the vacuum source (not shown) is activated. The most proximal expandable unit 301D may comprise a vacuum suction port 311, connectable to a vacuum source (not shown) and to a plurality of vacuum tubes 309 disposed on the outer surface of inflatable balloon 303. It is to be noted, that while the depiction herein illustrates a series of a plurality of expandable units 301A-301D organized in a linear manner, this arrangement is for illustrative purposes only and device 300 is designed to fit a variety of fistulas having different dimensions, as will be further elaborated in FIG. 3D. For illustrative purposes, the plurality of expandable units 301A-301D, in accordance with a non-limiting embodiment of the device 300 disclosed herein, are represented in FIGS. 3A-3B with-(FIG. 3A) and without (FIG. 3B) an outer expandable envelope 307, in the latter case demonstrating the flexible properties of the plurality of vacuum tubes 309, which are configured such that they correspond to the change in dimensions and shape of an inflatable balloon 303A-303D when a balloon is at least partially inflated or deflated.

In accordance with an embodiment of the present disclosure, device 300 may further comprise at least one connection port 302, being configured for connecting, for example, at least one expandable unit 301A to another expandable unit 301B provided in series wherein, when connected, permits fluid communication between consecutive expandable units. Specifically, when negative pressure is applied, fluids from the fistula and/or fistula environment are drained and pass on to the vacuum suction port 311 at the proximal portion of the most proximal expandable unit 301A, via the plurality of vacuum tubes 309 in a distal to proximal direction. In a non-limiting embodiment of device 300, the at least one connection port 302 may also be configured to accommodate at least one of endoscopic tube or inflation tube being configured and operable to at least partially in situ inflate or deflate an inflatable balloon 303 of any of the expandable units 301A-301D. The at least one connection port 302 may be connectable with endoscopic inlet 313, preferably, albeit not exclusively, disposed in the distal end portion of the distal most unit 301D of the present configuration.

In a preferred non-limiting embodiment of device 300 disclosed herein, and as shown in FIG. 3C, each of the plurality of expandable units 301A-301D is configured to be selectively at least partially in situ inflated or deflated independently one to the other to different dimensions, i.e., different volumes, to provide an overall external shape of the device 300 corresponding to an internal shape of fistulas of various sizes and dimensions. In this way, when inflated, device 300 is able to effectively contact as much of the endoluminal surface of the fistula as possible. Moreover, to ensure a modular and dynamic fit, each expandable unit 301A-301D is further configured to be selectively, discretely inflated or deflated; also, being further configured to be sequentially at least partially inflated with respect to another expandable unit being placed proximally to form a distal portion having an adjustable length being configured to be deployed in fistulas of different dimensions. Thus, the configuration of the device embodied in FIGS. 3A-3C, presents an embodiment of the disclosure that comprises four different sets of balloons 303A-303D, corresponding to the four expandable units 301A-301D. It is to be noted, that inflatable balloons 303A-303D of the present disclosure may each comprise various shapes, such as narrow cylinders, elliptical, wider cylinders, or a combination of cylinders of heterogeneous diameter, narrow in portions relatively close to the proximal end, and wider at the distal end.

As can be appreciated in FIG. 3D, to achieve a shape of the device 300 corresponding to fistulas of different shapes and dimensions, the at least one connection port 302 may be further configured to allow an angular orientation of at least one expandable unit with respect to another expandable unit, such that an outer shape of the plurality of the expandable units 301A-301D, when connected, may better correspond to the dimensions and the shape of the fistula. The angular orientation may enable providing a device having an external shape in a manner reminiscent of the organization of the earthworm's (phylum Annelida) “rings” or annuli, corresponding to the fistula's inner shape.

For improving healing processes of the fistula, device 300 is further configured to permit the gradual withdrawal of the at least one expandable unit 301A to facilitate gradual healing of the wound from the deeper, distal direction to the more superficial, proximal direction. This is to prevent the fistula wound from healing and closing in the superficial direction, while an active wound remains in the depth of the tissue. To achieve this, each expandable unit 301A-301D is configured to be sequentially deflated with respect to the expandable unit being placed distally to allow gradual removal of the device as the fistula heals and shrinks.

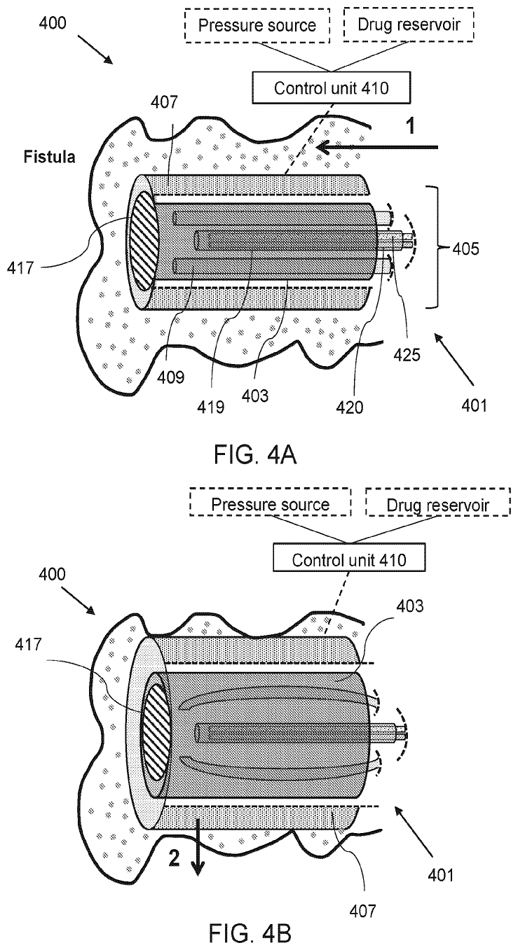

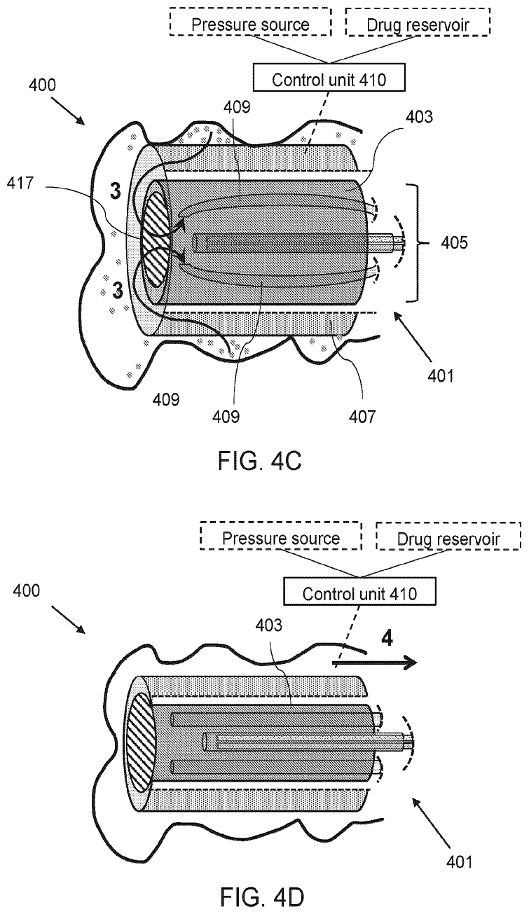

Reference will now be made to FIGS. 4A-4D, which illustrate a possible non-limiting embodiment of a device 400 for in situ endoscopic vacuum-assisted fistula-therapy. As can be appreciated in FIG. 4A, device 400 comprises at least one expandable unit 401, comprising an inflatable balloon 403 and an outer expandable envelope 407. In accordance with an embodiment of the present disclosure, device 400 may further comprise control unit 410 being connectable to a pressure source and being configured and operable to operate the device by applying vacuum in a constant or variable manner. The vacuum is applied via at least one vacuum tube shown in FIGS. 1-3 above to each at least one expandable unit separately. Control unit 410 may be configured and operable to apply a plurality of pulses of negative or positive pressures. The term “control unit” should be interpreted broadly, covering a computing/electronic utility including, inter alia, such utilities as data input and output modules/utilities, memory (i.e., non-volatile computer readable medium), and data processor module, as well as cloud computing-based system. The latter is a type of Internet-based computing that provides shared computer processing resources and data (such as servers, storage, and applications) to computers and other devices through the computer network (or communication network), such as the Internet.

In general, control unit 410 may be a processor, a controller, a microcontroller, or any kind of integrated circuit. The utilities of control unit 410 may thus be implemented by suitable circuitry and/or by software and/or hardware components including computer readable code configured for operating the device. Features within the scope of the present disclosure also include computer-readable media for carrying out or having computer-executable instructions, computer-readable instructions, or data structures stored thereon. Such computer-readable media may be any available media, which are accessible by a general-purpose or special-purpose computer system. Computer-readable media may include a computer program or computer application downloadable to the computer system over a network, such as a wide area network (WAN), e.g., Internet. In this description and in the following claims, a “control unit” is defined as one or more software modules, one or more hardware modules, or combinations thereof, which work together to perform operations on electronic data. The physical layout of the modules is not relevant. A computer system may include one or more computers coupled via a computer network. Likewise, a computer system may include a single physical device where internal modules (such as a memory and processor) work together to perform operations on electronic data.

Control unit 410 may be comprised of a processor embedded therein running a computer program or attached thereto. The computer program product may be embodied in one or more computer readable medium(s) having computer readable program code embodied thereon. The computer readable medium may be a computer readable signal medium or a computer readable storage medium. Computer program code for carrying out operations for aspects of the present disclosure may be written in any combination of one or more programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). These computer program instructions may be provided to the processor of a general-purpose computer, special purpose computer, or other programmable data processing apparatus to produce a machine, such that the instructions, which execute via the processor of the computer or other programmable data processing apparatus. The specified functions of the control unit can be implemented by special purpose hardware-based systems that perform the specified functions or acts, or combinations of special purpose hardware and computer instructions.

In accordance with some embodiments of the device, as demonstrated in FIG. 4, the vacuum is applied via an at least one vacuum tube 409, and in a manner similar to that illustrated in FIGS. 1-3 above, to each of the at least one expandable unit 401. Control unit 410 may be configured and operable to apply a plurality of pulses of negative or positive pressures. Furthermore, it may be configured to apply vacuum to each of the at least one expandable unit discretely.

As can be appreciated in FIG. 4A, inner core 405 of device 400 may comprise at least one open-bore sheath 419 being configured for accommodating at least one insertion channel 420, being configured and operable for at least one of temporary insertion, manipulation, or activation of at least one of (i) at least one surgical tool, or (ii) an immobilizer being configured and operable to attach device 400 to the fistula. The number of insertion channels is not limited and may be unified, hybrid, or otherwise incorporated together into a single (one), two, three, four, or five different channels. The surgical tools may be selected from at least one of manipulators, light, heat or electricity emitters, sensors, obstetrical forceps, trocar incision closure devices, electrodes and electrosurgical cables, cannulas and trocars, laparoscopic bipolar scissors and graspers, forceps and graspers, hooks and probes, knot pushers, needles and needle holders, retractors, scissors, suturing tools, probes, dissectors, or any combination thereof. The immobilizer as used herein, is selected from an anchoring means, locators, fixating means, sutures, patch, clamp(s), biocompatible adhesives, etc., including those shaped as inflatable balloons, or made of shape memory alloys (e.g., nitinol), anchors or studs of changeable (open-close, thin-wide) configuration. Additionally, or alternatively, inner core 405 may comprise at least one drug delivery tube 425 being configured and operable to allow passage of the drug therethrough and to administer at least one drug in a proximal to distal direction of the device 400. In this case, control unit 410 may be further connectable to a drug reservoir and may be configured and operable to administer a drug by applying a plurality of pulses of positive pressure e.g., via the drug delivery tube.

FIGS. 4A-4D demonstrate the different operational modes of the use of in situ vacuum-assisted fistula-therapy device 400. If required, the use comprises inserting an endoscope tube at a location at the distalmost portion of the device 400, via the open-bore sheath 119, which may comprise an endoscope inlet for affixing an endoscope tube (not shown). It is to be noted, that open-bore sheath 119, may optionally comprise at least one insertion channel 420 being configured and operable for at least one of temporary insertion manipulation or activation of at least one of (i) at least one surgical tool or (ii) an immobilizer being configured and operable to be attached to the fistula, and at least one drug delivery tube 425. The use comprises advancing the in situ vacuum-assisted fistula-therapy device 400, comprising at least one expandable unit 401, towards a fistula to be treated in a distal to proximal direction, as indicated by arrow 1. Then, in situ at least partially inflating the inflatable balloon 403, as shown in FIG. 4B, inflatable balloon 403 pushing at least a portion of the expandable outer envelope 407, in a direction as indicated by arrow 2, to contact at least partially the endoluminal surface of the fistula without applying shear force on the tissue. In accordance with an embodiment of the present disclosure, the inflatable balloon 403 may at least partially be inflated by passing/applying a fluid (e.g., saline, purified water, CO₂ gas, etc.) through an inflation or endoscope tube (not shown), accommodated within open-bore sheath 419, in a predefined volume, rate, and pressure, so that inflatable balloon 403 is shaped and having the corresponding dimensions of the fistula and/or of an area adjacent to the fistula.

As can be appreciated in FIG. 4C, following at least partial inflation of the inflatable balloon 403 a negative pressure is applied by use of control unit 410, in communication with a pressure source (not shown), through any of vacuum tubes 409, thereby draining fluids from the fistula cavity, passing through the outer expandable envelope 407, comprised preferably, as described above, of a porous material, towards the inner core 405, and then passing through any of vacuum tubes 409, towards the vacuum suction port (not shown). As can be appreciated in FIGS. 4A-4D, vacuum tubes 409 may have flexible properties, such that the outer shape of vacuum tube 409 corresponds to the shape of the outer surface of inflatable balloon 403 when it is at least partially inflated or deflated. Device 400 may also be configured to have each of the at least one expandable unit 401 configured to be sequentially at least partially inflated with respect to another expandable unit being placed proximally to form a distal portion having an adjustable length being configured to be deployed in fistulas of different dimensions (as demonstrated in detail in FIG. 3 above).

The control unit 410 may apply a vacuum in either a constant or variable manner over time via at least one of vacuum tubes 409. Additionally, or alternatively, control unit 410 may apply train of pulses over time. The pulses may be trains of both (i) pulses of negative pressure being either constant or variable; and (ii) at least one positive pressure. The application of such pulses enables to prevent from the outer expandable envelope 407, and consequently the at least one expandable unit 401, to be blocked by fluids (of varying viscosity; gel-like non-or less-flowing substances), or solids (blood clots), depicted in FIGS. 4A-4D as grey dots (illustrating a fluid with anticipated dissolved particles), drained from the fistula and to advance the device progressively in the fistula by operating a series of pulses.

Control unit 410 may also operate to apply a negative pressure wound therapy (NPWT) in a continuous mode. Additionally or alternatively, control unit 410 may also operate to apply an Intermittent pressure therapy (IPT). Generally, IPT results in faster wound healing and shortens the treatment time, but it often causes pain. Variable pressure therapy (VPT) provides a smooth transition between two different pressure environments, thereby maintaining the negative pressure environment throughout the therapy. Wound contraction and granulation tissue formation is more pronounced following IPT and VPT than continuous NPWT. As can be further appreciated in FIGS. 4A-4D, device 400 may preferably, albeit not exclusively, comprise a distal sealing element plug 417 for maintaining the effective negative and/or positive pressure environment throughout, when the pressure source is activated, operated by control unit 410.

In accordance with an embodiment of the present disclosure, control unit 410 is connectable to a drug reservoir and is configured and operable to administer a drug by applying a plurality of pulses of positive pressure. The drug is preferably passed through the inner core 405 of the expandable unit 401, comprising a drug delivery tube 425, that may be positioned within open-bore sheath 119 and configured and operable to allow passage of the drug therethrough.

As can be appreciated in FIG. 4D, when at least partially the fluid is drained from the fistula cavity, inflatable balloon 403 may be deflated and the device 400 gradually withdrawn from the wound site in the proximal to distal direction as indicated by arrow 4 at a suitable timepoint thereafter. For improving healing processes of the fistula, device 400 is further configured to permit the gradual withdrawal of the at least one expandable unit 401, wherein more than one expandable unit comprise the device (as exemplified in FIG. 3 above), to facilitate gradual healing of the fistula from the deeper, distal direction to the more superficial, proximal direction. This is to prevent the fistula wound from healing and closing in the superficial direction, while an active wound remains in the depth of the tissue. Thus, each of the at least one expandable unit is further configured to be sequentially deflated with respect to the expandable unit being placed distally to allow gradual removal of the device as the fistula heals and shrinks; ultimately eliminating the fistula and/or rendering it inactive.

One of ordinary skill in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

1. A device for in situ endoscopic vacuum-assisted fistula-therapy, the device comprising:
at least one expandable unit being configured and operable to be inserted into a fistula; said at least one expandable unit comprising:
a. an inflatable balloon;
b. an inner core located within the inflatable balloon;
c. an outer expandable envelope at least partially surrounding said inflatable balloon; and
d. at least one vacuum tube being disposed in between an outer surface of said inflatable balloon and the outer expandable envelope; wherein said outer expandable envelope is at least partially made of a porous material and is configured to allow passage of fluids from the fistula cavity towards said at least one vacuum tube; wherein said at least one vacuum tube is configured and operable to be connectable to a vacuum source to drain fluids having passed through the outer expandable envelope by applying a negative pressure.

2. The device of claim 1, wherein said inner core comprises at least one open-bore sheath being configured to be accommodated within.

3. The device of claim 2, wherein the at least one open-bore sheath is configured for at least one of accommodating an endoscope tube or inflating tube being couplable with the inflatable balloon and being configured and operable to at least partially inflate and deflate said inflatable balloon.

4. The device of any of the preceding claims, wherein said at least one expandable unit is configured to be inserted into the fistula while the inflatable balloon is in a deflated state, such that no shear force is applied on an endoluminal surface of the fistula.

5. The device of any of the preceding claims, wherein the inflatable balloon is configured to expand the outer expandable envelope when at least partially inflated.

6. The device of claim any of the preceding claims, wherein the inflatable balloon when at least partially inflated is configured to press an outer surface of the outer expendable envelope to contact an endoluminal surface of the fistula.

7. The device of any of the preceding claims, wherein said at least one vacuum tube is disposed on an outer surface of the inflatable balloon.

8. The device of any of the preceding claims, wherein said at least one vacuum tube has flexible properties, such that the outer shape of the at least one vacuum tube corresponds to the shape of the outer surface of said inflatable balloon.

9. The device of any of the preceding claims, further comprising at least one vacuum suction port being configured for connecting between a plurality of vacuum tubes of the same expandable unit.

10. The device of any of the preceding claims, further comprising at least one connection port being configured for connecting at least one expandable unit to another expandable unit provided in series; wherein, when connected, permits fluid communication between consecutive expandable units.

11. The device of claim 10, wherein said at least one connection port is configured to allow an angular orientation of at least one expandable unit with respect to another expandable unit, such that an outer shape of the plurality of the expandable units, when connected, corresponds to at least one of the dimensions and inner shape of the fistula.

12. The device of claims 10 to 11, wherein said at least one connection port is configured to accommodate at least one of endoscopic tube or inflation tube being configured and operable to at least partially in situ inflate or deflate an inflatable balloon of any of the expandable units.

13. The device of any of the preceding claims, wherein each expandable unit is configured to be in situ at least partially selectively inflated or at least partially selectively deflated independently one to the other.

14. The device of any of the preceding claims, wherein each expandable unit is configured to be in situ selectively at least partially inflated or deflated to different discrete dimensions to provide an external shape of the device corresponding to internal dimensions of a fistula.

15. The device of any of the preceding claims, wherein each expandable unit is configured to be sequentially at least partially inflated with respect to another expandable unit being placed proximally to form a distal portion having an adjustable length being configured to be deployed in fistulas of different dimensions.

16. The device of any of the preceding claims, wherein each expandable unit is configured to be selectively at least partially deflated to provide an external shape of the device corresponding to the internal dimensions of the fistula.

17. The device of any of the preceding claims, wherein each expandable unit is configured to be sequentially at least partially deflated with respect to the expandable unit being placed distally to allow gradual removal of the device as the fistula heals and shrinks.

18. The device of any one of the preceding claims, further comprising a control unit being connectable to a pressure source, said control unit being configured and operable to apply vacuum in a constant or variable manner via said at least one vacuum tube to each at least one expandable unit separately.

19. The device of claim 18, wherein said control unit is configured and operable to apply a plurality of pulses of negative or positive pressures.

20. The device of claims 18 to 19, wherein said control unit is connectable to a drug reservoir and is configured and operable to administer a drug by applying a plurality of pulses of positive pressure.

21. The device of claim 20, wherein the inner core further comprises at least one drug delivery tube configured and operable to allow passage of the drug therethrough.

22. The device of any of the preceding claims, wherein said inner core further comprises at least one insertion channel being configured and operable for at least one of temporary insertion manipulation or activation of at least one of (i) at least one surgical tool or (ii) an immobilizer being configured and operable to be attached to the fistula.

23. The device of any one of the preceding claims, wherein said at least one expandable unit has a length in the range of about 10 mm to 100 mm.

24. The device of any one of the preceding claims, wherein said outer expandable envelope is at least partially coated with at least one material being capable of at least one of accelerating healing or treating an infection.
